(19) United States
(12) Patent Application Publication
(10) Pub. No.: US 2019/0117132 A1
(43) Pub. Date: Apr. 25, 2019

(54) BIOFLUID SENSING DEVICE NUCLEOTIDE SENSING APPLICATIONS

(71) Applicant: Eccrine Systems, Inc., Cincinnati, OH (US)

(72) Inventors: Robert Beech, Cincinnati, OH (US); Gavi Begtrup, Cincinnati, OH (US); Benjamin Katchman, Cincinnati, OH (US)

(73) Assignee: Eccrine Systems, Inc., Cincinnati, OH (US)

(21) Appl. No.: 16/083,382

(22) PCT Filed: Mar. 9, 2017
(86) PCT No.: PCT/US17/21503

§ 371 Date: Sep. 7, 2018

Related U.S. Application Data
(60) Provisional application No. 62/306,380, filed on Mar. 10, 2016.

(51) Int. Cl. A61B 5/1477; A61B 5/00; A61B 5/145; A61B 5/117

(52) U.S. Cl. CPC A61B 5/1477; A61B 5/6898; A61B 5/746; A61B 5/117; A61B 5/1451

(57) ABSTRACT

The disclosed invention addresses an unmet need of biofluid analysis by utilizing the presence of certain nucleotides, e.g., microDNA, eccDNA, DNA fragments, microRNA, RNA fragments, and peptides, in a sample biofluid, such as eccrine sweat, apocrine sweat, blood, serum, saliva or tears, to perform a number of physiological sensing functions. Specifically, the present disclosure provides: (1) a device and method of determining whether a biofluid sensing device is being worn by a specific individual based on a measurement of nucleotides in a biofluid; (2) a device and method of recognizing at least one physiological disease or condition based on nucleotide concentrations, ratios, or trends in a biofluid.

### CROSS-REFERENCE TO RELATED APPLICATIONS

The present application claims priority to PCT/US17/21503, filed Mar. 9, 2017, and has specification that builds upon PCT/US15/55756, filed Oct. 15, 2015; and PCT/US16/36038, filed Jun. 6, 2016, the disclosures of which are hereby incorporated herein by reference in their entirety.

### BACKGROUND OF THE INVENTION

Despite the many ergonomic advantages of perspiration (sweat) compared to other biofluids (particularly in “wearable” devices), sweat remains an underutilized source of biomarker analytes compared to the established biofluids: blood, urine, and saliva. Upon closer comparison to other non-invasive biofluids, the advantages may even extend beyond ergonomics: sweat might provide superior analyte information. Several challenges, however, have historically kept sweat from occupying its place among the preferred clinical biofluids. These challenges include very low sample volumes (nL to μL), unknown concentration due to evaporation, filtration and dilution of large analytes, mixing of old and new sweat, and the potential for contamination from the skin surface. More recently, rapid progress in “wearable” sweat sampling and sensing devices has resolved several of the historical challenges. However, this recent progress has also been limited to high concentration analytes (μM to mM) sampled at high sweat rates (>1 nL/min/gland) found in, for example athletic applications. Progress will be much more challenging as biosensing moves towards detection of large, low concentration analytes (nM to pM and lower).

A primary goal of the disclosed invention is to provide decision support to a biofluid sensing device user that is informative at the level of the individual patient. A biofluid sensing patch worn on the skin and connected to a computer network via a reader device, such as a smart phone or other portable or stationary device, could aid in recognition of the physiological state of an individual, and relay crucial data about sweat rate, biofluid flow rate, or other physiological states. In certain settings, biofluid sensing devices may continuously monitor certain aspects of an individual's physiological state and communicate relevant information to a reader device or computer network, which would then compare collected data to threshold readings and generate notification messages to the individual, a caregiver, a work supervisor, or other biofluid sensing device user.

In particular, biofluid sensing devices hold tremendous promise for use in workplace safety, athletic, military, personal wellness and healthcare settings. Many of these applications may be enabled or enhanced through biofluid detection of biological oligomers, including nucleotides. Various short-strand nucleotides emerge in sweat, such as microDNA (miDNA), eccDNA, DNA fragments, microRNA (miRNA), RNA fragments, peptides, and others. These nucleotides perform various extracellular signaling functions, for example, miRNA appears to play a significant role in exosomic endocrine modulation and mediation of tissue crosstalk, which facilitates immune response, among other functions. They may also find their way into sweat through excretion, for example miRNA fragments from viral infections. miRNAs are small (around 22 nucleotides), noncoding RNA sequences that chiefly repress gene translations by interacting with certain sections of the gene post-transcription. Several potential uses of biofluid nucleotides, specifically miRNAs, as detected by a biofluid sensing device, are disclosed herein.

Many of the drawbacks and limitations of sweat as a medium for continuous biosensing can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sweat sensing technology into intimate proximity with sweat as it is generated.

### DEFINITIONS

Before continuing with the background, a variety of definitions should be made, these definitions gaining further appreciation and scope in the detailed description and embodiments of the present invention.

As used herein, “sweat” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

As used herein, “biofluid” may mean any human biofluid, including, without limitation, sweat, interstitial fluid, blood, plasma, serum, tears, and saliva.

“Biofluid sensor” means any type of sensor that measures a state, presence, flow rate, solute concentration, solute presence, in absolute, relative, trending, or other ways in a biofluid. Biofluid sensors can include, for example, potentiometric, amperometric, impedance, optical, mechanical, antibody, peptide, aptamer, or other means known by those skilled in the art of sensing or biosensing.

“Analyte” means a substance, molecule, ion, or other material that is measured by a biofluid sensing device.

“Measured” can imply an exact or precise quantitative measurement and can include broader meanings such as, for example, measuring a relative amount of change of something. Measured can also imply a binary measurement, such as ‘yes’ or ‘no’ type measurements.

“Chronological assurance” means the sampling rate or sampling interval that assures measurement(s) of analytes in biofluid in terms of the rate at which measurements can be made of new biofluid analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5 to 30 minute lag time between analytes in blood emerging in interstitial fluid), but the resulting sampling interval (defined below) is independent of lag time, and furthermore, this lag time is inside the body, and therefore, for chronological assurance as defined above and interpreted herein, this lag time does not apply.

“Analyte-specific sensor” means a sensor specific to an analyte and performs specific chemical recognition of the analytes presence or concentration (e.g., ion-selective electrodes, enzymatic sensors, electro-chemical aptamer based sensors, etc.). For example, sensors that sense impedance or conductance of a fluid, such as sweat, are excluded from the definition of “analyte-specific sensor” because sensing impedance or conductance merges measurements of all ions in sweat (i.e., the sensor is not chemically selective; it provides an indirect measurement). Sensors could also be optical, mechanical, or use other physical/chemical methods which are specific to a single analyte. Further, multiple sensors can each be specific to one of multiple analytes.

“EAB sensor” means an electrochemical aptamer-based biosensor that is configured with multiple aptamer sensing elements that, in the presence of a target analyte in a biofluid sample, produce a signal indicating analyte capture, and which signal can be added to the signals of other such sensing elements, so that a signal threshold may be reached that indicates the presence of the target analyte.

“Docked aptamer EAB sensor” means an EAB sensor that employs docking strategies to connect analyte capture complexes with the sensor electrode, as disclosed in PCT/U.S. Ser. No. 18/39274, filed Jun. 25, 2018, which is hereby incorporated by reference in its entirety.

“Aptamer” means a molecule that undergoes a conformation change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function.

“Biofluid sensor data” means all the information collected by biofluid sensing device sensor(s) and communicated to a user or a data aggregation location.

“Correlated aggregated biofluid sensor data” means biofluid sensor data that has been collected in a data aggregation location and correlated with outside information such as time, temperature, weather, location, user profile, other biofluid sensor data, or any other relevant data.

“Analyte data signature” means a known set of analyte levels, ratios, or concentration trends that is correlated with a specific individual within a probability range. The analytes measured are comprised of biofluid nucleotides, including miRNAs.

“Identification Metric” means one of the various identification-related readings that may be used by a biofluid sensing device to indicate within a certain probability that a target individual is wearing the device. These metrics include, without limitation, sweat analyte data metrics, proxy identification metrics, communication/location metrics, or other metrics.

“Identification Profile” means a profile composed of at least one identification metric and associated with an individual for use in calculating an identification probability estimate.

“Nucleotide Signature” means a measurement of at least one nucleotide-based identification metric and associated with an individual for use in calculating an identification probability estimate.

“Identification probability estimate” means the calculated probability that a person wearing a biofluid sensing device is a target individual based on a comparison of identification metrics with known data about the target individual.

“Operation and compliance alert” means a message generated by the biofluid sensing device and relayed to a user when an operation and compliance reading indicates a wearer identification status.

“Upregulated” means a concentration of miRNA or other nucleotide that is elevated in serum, a body tissue, sweat, or other body fluid relative to a normative baseline concentration.

“Downregulated” means a concentration of miRNA or other nucleotide that is lower in serum, a body tissue, sweat, or other body fluid relative to a normative baseline concentration.

This has served as a background for the disclosed invention, including background technical invention needed to fully appreciate the disclosed invention, which will now be summarized.

### SUMMARY OF THE INVENTION

The disclosed invention addresses an unmet need involving biofluid analysis by utilizing the presence of certain nucleotides in eccrine and or apocrine sweat, or other biofluid, to perform a number of physiological sensing functions. Specifically, the present disclosure provides: (1) a device and method of determining whether a biofluid sensing device is being worn by a specific individual based on a measurement of nucleotides in a biofluid; (2) a device and method of recognizing at least one physiological disease or condition based on nucleotide concentrations or ratios in a biofluid.

### BRIEF DESCRIPTION OF THE DRAWINGS

The objects and advantages of the disclosed invention will be further appreciated in light of the following detailed descriptions and drawings in which:

FIG. 1 is a generic representation of a device for carrying out the disclosed invention including a mechanism for analyzing biofluid sensor data on a singular, continuous, or repeated basis.

FIG. 2 is an example representation of at least a portion of a disclosed device, capable of detecting nucleotides in a biofluid sample.

FIG. 3 is an example flow chart representation of at least a portion of a disclosed method, including a process for identifying a wearer based on nucleotide biofluid concentrations or ratios.

FIG. 4 is an example flow chart representation of at least a portion of a disclosed method, including a process for determining whether a wearer has a physiological condition based on nucleotide biofluid concentrations or ratios.

### DETAILED DESCRIPTION OF THE INVENTION

The disclosed invention provides devices and methods for using biofluid-carried nucleotides, e.g., miRNA molecules, to identify a biofluid sensing device wearer, or to recognize a disease or physiological condition based on a profile of upregulated or downregulated nucleotide molecules in the biofluid.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

Certain embodiments of the disclosed invention show sensors as simple individual elements. It is understood that many sensors require two or more electrodes, reference electrodes, or additional supporting technology or features which are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referred to by what the sensor is sensing, for example: a biofluid sensor; an impedance sensor; a sample volume sensor; a sample generation rate sensor; and a solute generation rate sensor. Certain embodiments of the disclosed invention show sub-components of what would be sensing devices with more sub-components needed for use of the device in various applications, which are obvious (such as a battery), and for purposes of brevity and focus on inventive aspects, such components are not explicitly shown in the diagrams or described in the embodiments of the disclosed invention. As a further example, many embodiments of the disclosed invention could benefit from mechanical or other means known to those skilled in wearable devices, patches, bandages, and other technologies or materials affixed to skin, to keep the devices or sub-components of the skin firmly affixed to skin or with pressure favoring constant contact with skin or conformal contact with even ridges or grooves in skin, and are included within the scope of the disclosed invention.

The detailed description of the present invention will be primarily, but not entirely, limited to devices, methods and sub-methods using wearable biofluid sensing devices. Therefore, although not described in detail here, other essential steps which are readily interpreted from or incorporated along with the present invention shall be included as part of the disclosed invention. The disclosure provides specific examples to portray inventive steps, but which will not necessarily cover all possible embodiments commonly known to those skilled in the art. For example, the specific invention will not necessarily include all obvious features needed for operation. Several specific, but non-limiting, examples can be provided as follows. The invention includes reference to the article in press for publication in the journal IEEE Transactions on Biomedical Engineering, titled “Adhesive RFID Sensor Patch for Monitoring of Sweat Electrolytes”; the article published in the journal AIP Biomicrofluidics, 9 031301 (2015), titled “The Microfluidics of the Eccrine Sweat Gland, Including Biomarker Partitioning, Transport, and Biosensing Implications”; as well as PCT/US16/36038, and U.S. Provisional Application No. 62/327,408, each of which is included herein by reference in their entirety. Techniques for concentrating a biofluid sample are disclosed in PCT/US16/58356, and U.S. Provisional Application No. 62/457,604, which are also hereby incorporated herein by reference in their entirety. In addition, the invention may include electrochemical aptamer-based (“EAB”) sensors, such as are disclosed in U.S. Pat. Nos. 7,803,542 and 8,003,374, and which represent a stable, reliable bioelectric sensor that is sensitive to the target analyte in biofluid, while being capable of multiple analyte capture events during the sensor lifespan.

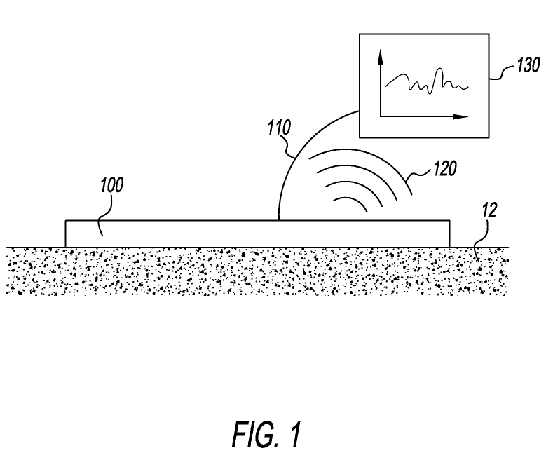

With reference to FIG. 1, a representative biofluid sensing device 100 to which the present disclosure applies is placed on or near skin 12. The biofluid sensing device may be connected to skin or regions near skin through microfluidics or other suitable techniques, such as an adhesive layer (not shown). Adhesives can be pressure sensitive, liquid, tacky hydrogels, which promote robust electrical, fluidic, and iontophoretic contact with skin. The device 100 is in wired communication 110 or wireless communication 120 with a reader device 130, which could be a smart phone or portable electronic device, or for some devices, the device 100 and reader device 130 can be combined. Communication 110 or 120 m and could bay be constant, periodic, or a simple one-time data download from the device once the device has completed its measurements of biofluid. The device includes at least one analyte-specific sensor, e.g., an ion-selective electrode sensor, or an EAB sensor.

The disclosed biofluid sensing device could also include a plurality of secondary sensors to improve detection of biofluid analytes, including a reference electrode, a pH sensor, a temperature sensor, a skin impedance sensor, a capacitive skin proximity sensor, a heart rate monitor, and an accelerometer. Many of the auxiliary features of the invention may, or may not, require other aspects of a biofluid sensing device, including two or more counter electrodes, reference electrodes, or additional supporting technology or features, which are not captured in the description herein, such as an onboard flash memory (i.e., 1MB minimum), a real-time clock, Bluetooth' or other communications hardware, and a multiplexer to process a plurality of sensor outputs.

The disclosed biofluid sensing device also includes computing and data storage capability sufficient to operate the device, which incorporates the ability to conduct communication among system components, to perform data aggregation, and to execute algorithms capable of generating notification messages. The device may have varying degrees of onboard computing capability (i.e., processing and data storage capacity). For example, all computing resources could be located onboard the device, or some computing resources could be located on a disposable portion of the device and additional processing capability located on a reusable portion of the device. Alternatively, the device may rely on portable, fixed or cloud-based computing resources.

The biofluid sensing device's data aggregation capability may include collecting all of the biofluid data generated by biofluid sensing devices and communicated to the device. The aggregated biofluid sensor data could be de-identified from individual wearers, or could remain associated with an individual wearer. Such data can also be correlated with outside information, such as the time, date, medications, drug sensitivity, medical condition, activity performed by the individual, motion level, fitness level, mental and physical performance during the data collection, body orientation, the proximity to significant health events or stressors, age, sex, weight, health history, or other relevant information. The reader device or companion transceiver can also be configured to correlate speed, location, environmental temperature or other relevant data with the biofluid sensor data. The data collected could be made accessible via secure website portal to allow device users to perform safety, compliance and/or care monitoring of target individuals. The biofluid sensor data monitored by the user includes real-time data, trend data, or may also include aggregated biofluid sensor data drawn from the system database and correlated to a particular user, a user profile (such as age, sex or fitness level), weather condition, activity, combined analyte profile, or other relevant metric. Trend data, such as a target individual's hydration level over time, could be used to predict future performance, or the likelihood of an impending physiological event. Such predictive capability can be enhanced by using correlated aggregated data, which would allow the user to compare an individual's historical analyte and external data profiles to a real-time situation as it progresses, or even to compare thousands of similar analyte and external data profiles from other individuals to the real-time situation. Biofluid sensor data may also be used to identify wearers that are in need of additional monitoring or instruction, such as the need to drink additional water, or to adhere to a drug regimen.

Sweat is known to contain a large number of compounds that could be used to indicate an individual's physiological state. In general, however, determining an individual's physiological state is a significant challenge. Not only is every individual different in terms of how a physiological state may present, but even a simple physiological state or disorder is a complex set of biological processes that does not readily lend itself to reduction. Consequently, a definitive diagnosis of a physiological condition often is not possible. Rather, it may be necessary to classify individuals according to phenotypes or susceptibilities that indicate the mode in which a physiological state is likely to manifest in those individuals. These phenotypes may be indicated by analyte signatures that emerge in sweat. Among the most common substances found in sweat are the following: Na⁺, Cl⁻, K⁺, ammonium (NH₄⁺), urea, lactate, bicarbonate, glucose, serine, glycerol, cortisol, and pyruvate. In addition to one or more of these common sweat solutes, each physiological condition may also have particular sweat analytes that will prove informative for indicating that physiological state.

To date, there have been few studies linking sweat analyte measurements to physiological states. Among these are studies linking increased sweat chloride levels with cystic fibrosis, or a spike in chloride levels with ovulation. It will be necessary to build data across multiple individuals correlating physiological context, risk factors, phenotypes, and so on, with biofluid sensor readings. By this means, a discernible sweat analyte signature can be associated with a given physiological state for an individual or group of individuals.

Further, translation of analyte concentrations and ratios to meaningful physiological information must account for a number of variabilities unrelated to differences in concentrations. For example, sweat concentrations of analytes relative to blood or plasma concentrations are known to vary depending on sweat rate, the body location from which a sample is taken, kidney or liver disease or function, external temperatures, and other factors. To develop meaningful physiological information, algorithms and interpretive techniques will reflect how the various analyte signatures change in response to these variabilities. For example, sweat rate can inform many biofluid sensing device applications. For prolonged, continuous, or semi-continuous sweat analyte measurement, meaningful readings can only be taken at chronologically assured sweat sampling rates or intervals to ensure measurements are taken on fresh sweat as it emerges from a device wearer's skin. In addition, for larger molecules (e.g., proteins) that do not readily partition into sweat, sweat rate will likely be a critical component of a meaningful biofluid sample concentration measurement. The sweat concentrations of such analytes are expected to be below the sensitivity levels of most wearable sensor technologies. Therefore, biofluid samples may need to be concentrated in order to reach analyte molarities that are in the detectable range. To translate detected molarities into physiologically meaningful information, some applications will require sweat rate to back-calculate the measurements into an unconcentrated analyte molarity.

Within this context, the disclosed invention provides for the use of biofluid sensing devices that are configured to measure biofluid nucleotide levels in order to provide physiological information about the device wearer. Certain miRNA molecules, including those known as circulating miRNA, actively or passively emerge in blood, interstitial fluid, and eccrine and apocrine sweat. These molecules are typically bound with protective complexes, most commonly the following: (1) exosomes and other microvesicles, (2) Ago-2-miRNA complexes, and (3) HDL-mediated transport. See, e.g., Shaffer, J., et al., “miRNA profiling from blood,” Qiagen, Inc. Whitepaper, 2012. These protective complexes add a layer of complexity to nucleotide sensing, since biorecognition sensor elements such as aptamers, will not be designed to bind with the protective complexes, but instead will bind with the target nucleotide molecule itself. Another difficulty is that unbound miRNAs have a relatively short lifespan in sweat or other biofluids, because RNases seek out and destroy them. This makes direct detection of miRNAs in their unbound state more difficult. In addition, sweat concentrations of these nucleotides are expected to be below the detection levels of most known sensor technologies.

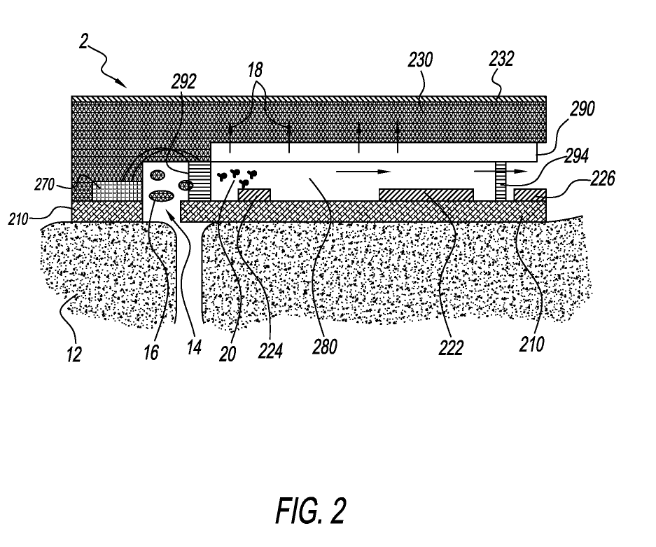

One solution to these challenges disclosed herein is to configure the biofluid sensing device to lyse protective complexes, filter out unwanted solutes, and concentrate the biofluid sample with respect to the target nucleotide, before the biofluid sample reaches the EAB sensor. For example, with reference to FIG. 2, a device 2 is placed on skin 12. The device comprises an adhesive layer (not shown), a fluid impermeable substrate 210, a power controller 270, an EAB sensor 222, and a concentration channel 280. A sweat sample 14 with nucleotide protective complexes 16, such as exosomes, enters the device, and moves toward a lysing membrane 292 that gates the entrance of the concentration channel 280. The lysing membrane 292 is a selectively porous membrane, such as a dialysis or osmosis membrane, that is impermeable to miRNA exosomes (30 to 120 nm diameter), or other conveyances for nucleotides, e.g., microvesicles (50 to 1,000 nm diameter), extracellular vesicles (30 to 2,000 nm diameter), and apoptotic bodies (50 to 2,000 nm diameter) and larger solutes, but permeable to unbound miRNA molecules (≈7.3 kDa). Because of the wide size variability for such protective complexes, membrane pore size may require adjustment to optimize collection of the target nucleotide molecule for a particular device application.

The lysing membrane further has available electrical current that is applied by the power controller 270. The collected protective complexes 16 in the sweat sample are too large to pass through, and instead become lodged on, the lysing membrane 292. The power controller 270 periodically applies current to the lysing membrane 292, which lyses the protective complexes. Such applied current causes the nucleotide molecules 20 to separate from the protective complexes and pass through the lysing membrane 292. Some embodiments do not apply current to the lysing membrane 292, but instead the upstream side of the membrane is coated with a detergent (not shown) that lyses the protective complexes. In some embodiments, the lysing membrane is permeable to unbound miRNA molecules (≈7.3 kDa), but is impermeable to RNase molecules (≈13 to 17 kDa), thus preserving the miRNA in its unbound state.

At this point, the nucleotide molecules 20 proceed through the concentration channel 280, where water and other solutes 18 pass through the concentrating membrane 290, which concentrates the biofluid sample with respect to the target nucleotide molecules. The concentrating membrane is a similar material to the lysing membrane, and is at least permeable to water and impermeable to the target nucleotide molecule.

In fluid communication with the concentrating membrane is a pump 230, which is configured facilitate either wicking or osmotic flow, and is a hydrogel, textile, salt, polyelectrolyte solution, or desiccant, such as MgSO₄. Depending on the application, the target analyte may be concentrated at least 10×, 100×, or 1000× higher than its original concentration in biofluid. As fluid leaves the sample, the target nucleotide molecules 20 continue through the concentration channel, and are detected by EAB sensor 222. The device may also include an optional post-sensor membrane 294. Some embodiments may also include secondary sensors 224, 226, which can be located inside 224 or outside 226 the concentration channel. Some embodiments also include a fluid impermeable protective layer 232.

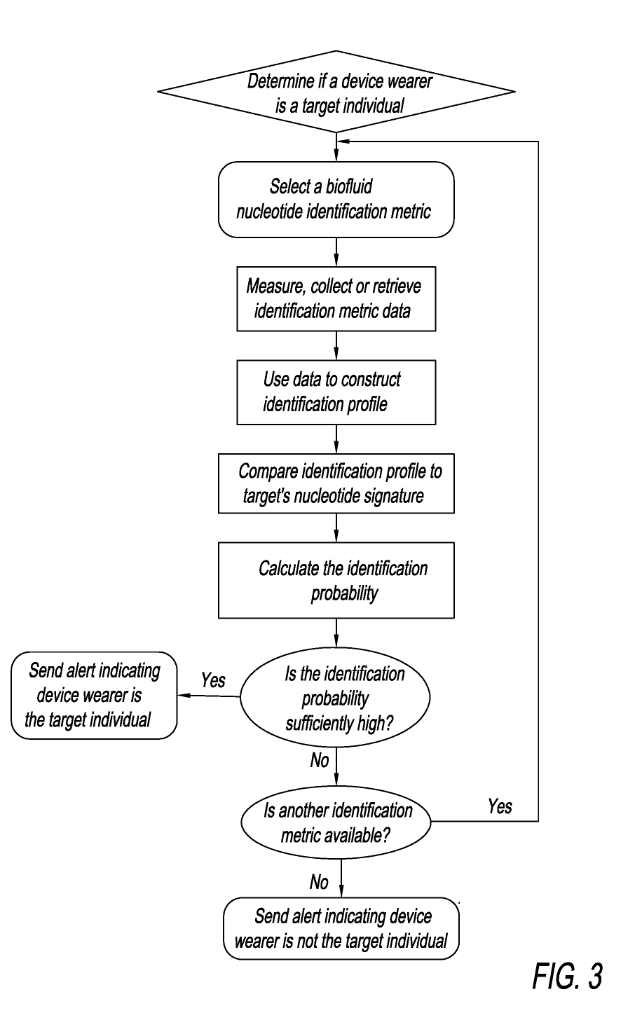

The use of the techniques disclosed herein may allow a biofluid sensing device to function as a biometric device. This capability enables the device to provide access to authenticated or high security systems. In addition, many other biofluid sensing device applications require confirmation that a target individual is wearing the biofluid sensing device. Therefore, in a preferred embodiment of the disclosed invention, the biofluid sensing device described above may determine the identity of a device wearer, or determine if a device wearer is a target individual, based on the biofluid detection of nucleotides such as miRNA. Example steps of the method for identifying a wearer based on biofluid detection of nucleotide molecules are summarized in FIG. 3.

The biofluid sensing device would take readings on a selected Identification Metric, would use those readings to develop an Identification Profile, and would then compare that profile to a Nucleotide Signature used for the target individual. Based on this comparison, the device would calculate an Identification Probability Estimate characterizing the probability that the wearer is the target individual. Several nucleotide-based identification metrics are available for use with a biofluid sensing device, based on individual differences in nucleotide concentrations and ratios that emerge in sweat. This nucleotide signature may comprise all or part of the target individual's identification signature.

The concentrations of different miRNA strands, and/or other biological oligomers in sweat or other biofluid, or the comparative ratios of such oligomers, or the ratios of such oligomers to other analytes, may be a strong indicator of identity, and thus can serve as a powerful Identification Metric for use by a biofluid device. To create an individual Nucleotide Signature, the device may be configured to measure specific nucleotide concentrations, trends or ratios that have been correlated to a target individual. For example, each individual may have a number of miRNA types that emerge in sweat, or that emerge in certain concentrations or ratios, etc. A biofluid sensing device may be configured to track a plurality of (e.g., eight) miRNA types in sweat designed to provide identification within a desired probability. An individual may possess none, all, or some fraction of the tracked miRNA types, and can thus be categorized by the device. In other embodiments, a target individual may undergo screening during which specific miRNA types, concentrations, or ratios are associated with that individual. Subsequently, devices may be configured to measure those specific miRNA types. In use, the device would collect measurements on the chosen miRNA types, and would incorporate those measurements into an Identification Profile. The device would then compare the measured Identification Profile with the appropriate Nucleotide Signature, and calculate an Identification Probability Estimate for the device wearer.

In another embodiment, nucleotide information may be used to identify a person within a reasonable probability without developing an individualized Nucleotide Signature. Some such embodiments may simply construct a profile of nucleotide concentrations, ratios or trends that are known to be generally applicable in identifying individuals with a desired level of certainty. Other embodiments may select general characteristics of the target individual, such as age, sex, hair color, eye color, height, or other characteristics, that are correlated with a certain Nucleotide Signature. For example, miRNA may be expressed in sweat in concentrations or ratios that can be correlated with the individual's phenotype, or physical characteristics. Once suitable characteristics have been identified, the user can select a device that is configured to measure the appropriate nucleotide concentrations or ratios that correspond to the selected features. While identification of a target individual by this method may not be as exact as using an individualized Nucleotide Signature, it may be sufficient for a number of applications.

If the desired certainty about the wearer's identity has not been reached after measuring a first identification metric, and if the device has another identification metric available, the device will measure another identification metric and calculate a new identification probability estimate. The process would continue until either the device has exhausted all of its available identification metrics, or the wearer has been positively or negatively identified as the target individual with sufficient certainty for the application. The device would then send an alert indicating whether or not the wearer is the target individual.

The biofluid sensing device may also communicate with other biometric scanners, such as a fingerprint or retina scanner, or other wearable fitness devices known in the art, such as an accelerometer, gait analysis sensor, heart rate monitor, impedance respiratory monitor, sensors measuring electrodermal activity, such as galvanic skin response, pulse oximetry, and others, to improve confidence in the identification results reached by both systems.

Using an Identification Probability Estimate or Nucleotide Signature with an indication that a biofluid sensing device is being worn, combines two powerful security paradigms: identification of the individual, and positive association of the device with that individual. Some embodiments, therefore, include sensors capable of detecting skin contact, such as skin impedance sensors, or a capacitive skin proximity sensor. Since the biofluid sensing device must be worn, and the device has the capability to determine whether it has been removed, the disclosed biofluid sensing device becomes a much more powerful identification device than one that an individual simply carries, such as a fob or key. By providing confidence that a biofluid sensing device is in skin contact with a target individual, the device prevents deceptive acts, such as wearing the device for an initial period and then simply removing it or passing it to another individual.

In addition to identifying a wearer, the disclosed invention assists in providing useful information related to the role of miRNA in the body. Because of their role in exosomic endocrine modulation, direct tissue interaction, and as excreted waste products, the presence of certain miRNAs in sweat or other biofluid may be indicative of several medical or other physiological conditions.

While the potential applications of a nucleotide-based biofluid sensing device are numerous, there are significant challenges to overcome in order to develop concentration data on sweat nucleotides. For instance, if concentration techniques are used to detect nucleotides, then the device will need to back-calculate the original nucleotide molarity. Additionally, the concentration of some nucleotides may be informative as to physiological condition only based on their upregulation or downregulation relative to a baseline concentration. For example, an individual may have a “normal” concentration of miRNAs in serum, which becomes upregulated in serum during an inflammatory immune response. However, an upregulated concentration of the miR-155 would be difficult to discern without a baseline or normalization measurement. In other cases, the amount of upregulation may be so small relative to the total concentration, that such change is not easily resolvable by the biofluid sensing device. For example, if the normal miR-155 concentration were 3 pmol, and the upregulated concentrated were 10 pmol, the device's EAB sensor suite would have to be quite sensitive to register such a change.

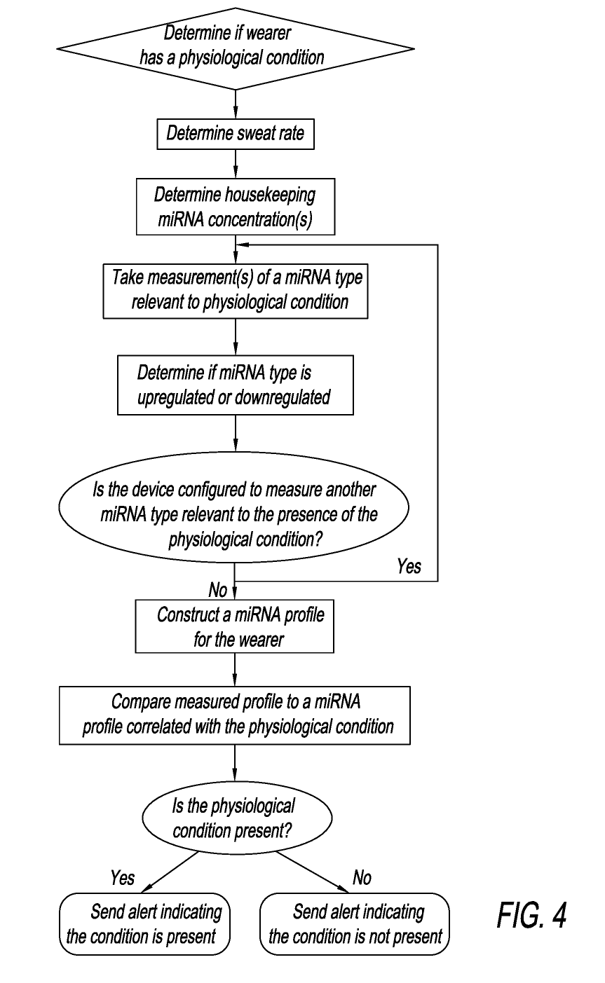

One potential solution is the use of certain miRNA types, e.g., miR-16 and miR-93, known as “housekeeping” miRNAs, which appear in serum at relatively predictable concentrations across broad populations. A biofluid sensing device configured to measure concentrations of miRNA in sweat may therefore measure the concentrations of such housekeeping miRNAs to normalize the sweat sample and determine whether a measured miRNA was upregulated or downregulated. Alternately, the device could normalize miRNA measurements by estimating plasma volume, which may be accomplished by determining a wearer's hydration level or tracking water loss with a sweat sensor, or by using body impedance measurements. In addition to normalization of the sweat sample, because some miRN As tend to be released into intracellular fluid locally near their target tissues, biofluid sensing device placement on a wearer's body may depend at least in part upon the location of the target tissue. For example, a biofluid sensing device configured to detect miRNA markers of sun damage in a wearer's skin may need to be placed in proximity to the suspected sun damage, e.g., on skin displaying increased redness from sun exposure. Additionally, because a particular miRNA may have a number of different targets (usually around 400), a single raised or lowered miRNA concentration, standing alone, is unlikely to allow a definitive determination about the presence of a condition. Therefore, it will likely be necessary to consider measured miRNA levels in the context of other relevant facts, for example, the concentration levels of other miRNA molecules, or the type of physical activity undertaken by the wearer, or known facts about the wearer's physical condition. Example steps of the method for identifying a physiological condition based on sweat detection of miRNA are summarized in FIG. 4.

One application for miRNA biofluid sensing is assessing a wearer for chronic inflammation [McDonald, et al., “Functional significance of macrophage-derived exosomes in inflammation and pain,” Pain 155:1527-1539, 2014; Alexander, M., et al., “Exosome-delivered microRNAs modulate the inflammatory response to endotoxin,” Nat. Commun. 6:7321, 2015; Ono, et al., 2014; Pulliam, L., Gupta, A., “Modulation of cellular function through immune-activated exosomes,” DNA Cell Biology 34:459-463, 2015], including neuroinflammation [Gupta A., Pulliam L., “Exosomes as mediators of neuroinflammation,” J. Neuroinflammation 11:68, 2015.] For example, it has been shown that miRNAs produced by T follicular helper cells help to regulate inflammation. In mice, miR-146a acts to suppress inflammation, while miR-155 tends to cause inflammation [See Hu, et al., “MiR-155 Promotes T Follicular Helper Cell Accumulation,” Immunity 41, 605-619, Oct. 16, 2014. Therefore, a biofluid sensing device that detected the concentrations or ratios of these miRNAs, (or other nucleotides performing similar functions in humans) could determine the presence, location, or extent of chronic, low-grade inflammation.

Another set of conditions in which miRNA plays a significant role is cancer. Studies indicate that certain tumor-promoting miRNAs, such as miR-21, miR-155, miR-17-92, contribute to the growth and proliferation of tumors across many different body tissues, while tumor-suppressant miRNAs, like miR-143, miR-145, miR-203, generally tend to hamper the growth of tumors. Further, many types of cancer are also associated with a general decrease in the amount of miRNA of all types within the body. Micro RNAs also influence the immune response to cancer, which contributes to decreases in tumor size. [Filipazzi, et al., “Recent advances on the role of tumor exosomes in immunosuppression and disease progression,” Semin. Cancer Biology 22:342-349, 2012.] The let-7 family of miRNAs regulates the expression of RAS proteins, which play a role in cell growth, thus these miRNAs may contribute to the suppression of cancer cell growth. [Sassen, et al., 2008]. Biofluid sensing devices accordingly may be configured to detect a plurality of miRNA concentrations in sweat that the device can then analyze to determine a probability of the existence of some form of cancer in the device wearer. For example, a device configured to detect a global decrease in miRNAs might serve as a screening device for the presence of cancer in the device wearer's body.

In addition to general cancer indications that may be discernable from miRNA concentrations or ratios in sweat, literature in the field indicates that many types of cancer may be correlated to a specific miRNA profile. For example, please see the following table, which shows miRNAs that are upregulated (i.e., found in higher-than-baseline serum concentrations) or downregulated (i.e., found in lower-than-baseline serum concentrations) for persons diagnosed with certain cancers:

| Cancer type | Relevant miRNA | Regulation |
|---|---|---|
| lymphoma | miR-155; miR-17-92 | up |
| leukemia | miR-15; miR-16; miR-92a | down |
| squamous cell | miR-184, miR-21 | up |
| carcinoma | | |
| squamous cell | miR-205 | down |
| carcinoma | | |
| prostate | miR-141; miR-375 | up |
| breast | miR-21, miR-125b | up |
| lung | miR-25; miR-223 | up |
| pancreas | miR-210, miR-200a and miR-200b | up |
| colon/rectum | miR-17-3p; miR-92 | up |
| ovary | miR-21; miR-29a; miR-92; miR-93; | up |
| | miR-126 | |

Therefore, in addition to general cancer screening, a biofluid sensing device may be configured to detect miRNA patterns indicative of a specific type of cancer. For example, a biofluid sensing device configured to screen a device wearer for colorectal cancer could have a suite of aptamer-based sensors capable of detecting upregulated concentrations of miR-17-3p and miR-92a in the wearer's sweat or other biofluid. Similarly, a device could screen for lymphomas by measuring the concentrations of the miR-17-92 group of miRNAs, or ratios of this group of miRNAs to other miRNAs, nucleotides, or analytes.

MicroRNAs also appear to have a significant role in general immune response. For example, miR-150 functions to repress the maturation of B lymphocytes, or B cells. Therefore upregulated miR-150 in the body would tend to repress immune response. The body's innate, or non-specific, immune response may be partially regulated through miRNAs, for example, downregulation of miR-155 in dendritic cells is associated with impaired antigen production and lower inflammatory response, while miR-155 upregulation is associated with increased inflammation. MiR-146a is similarly positively correlated with inflammatory response, while miR-125b is negatively correlated with inflammation (downregulated miR-125b is associated with increased inflammation). A biofluid sensing device configured to assess innate immune response may therefore be configured to detect miR-155, miR-146a, and miR-125b, and placed on the skin of a wearer. If the device detected upregulated sweat concentrations of miR-155 and miR-146a, along with downregulated sweat concentrations of miR-125b, the device could indicate that an aggressive inflammatory response was underway in the wearer's body.

The function of an individual's adaptive, or specific, immune response may also manifest in a discernible miRNA profile. For example, miR-155 plays a critical role in humoral and cellular immune response, as well as immune system adaptive memory. Therefore, low levels of miR-155 can be correlated with reduced adaptive immune response capability. The miR-17-92 group of miRNAs has a significant role in promoting Follicular B helper T cell development, while miR-181 promotes B cell development. A biofluid sensing device configured to measure sweat concentrations of these miRNAs could therefore indicate the function of a wearer's adaptive immune response.

Alternatively, since viral miRNA remnants appear in sweat after the body destroys a virus, and metabolic breakdown products of the invading virus may also appear in sweat, the biofluid sensing device could be configured to detect such remnants. [See Turchinovich, A., et al., “Characterization of extracellular circulating microRNA,” Nucleic Acids Research, doi:10.1093/nar/gkr254, 2011 (discussing the stability of viral miRNA/Ago2 complexes in plasma and reasoning that such complexes should persist in extracellular space after the death of the infected host cell).] Therefore, the device, for example, could be configured to detect miRNA fragments from the 6 top viruses for the season to determine whether a wearer had suffered from common illnesses. There may also be other miRNA waste products excreted in sweat that could be informative of body condition.

MiRNAs may also play a role in autoimmune disorders. [Mobergslien, A., Sioud, M., Exosome-derived miRNAs and cellular miRNAs activate innate immunity,” J. Innate Immun. 6: 105-110, 20141 As is the case with various types of cancer, literature in the field indicates that particular autoimmune disorders may also be correlated to a miRNA profile. For example, please see the following table, which shows miRNAs that are upregulated or downregulated for persons diagnosed with certain autoimmune disorders:

| Disorder | Relevant miRNA | Regulation |
|---|---|---|
| systemic lupus | miR-101; miR-125a; miR-146a; miR-142 | down |
| erythematosus | | |
| systemic lupus | miR-155; miR-21 | up |
| erythematosus | | |
| allergies | miR-21; miR-126; miR-146; miR-223 | up |
| allergies | miR-375 | down |
| rheumatoid | miR-155; miR-146a; miR-132; miR-16 | up |
| arthritis | | |
| rheumatoid | miR-142 | down |
| arthritis | | |
| Crohn's | miR-196 | up |
| colitis | miR-21 | up |
| psoriasis | miR-146a; miR-203; miR-31; miR-21 | up |

MiRNAs may also be informative in cases of organ transplant rejection. [See Agarwal, et al., “Regulatory T cell-derived exosomes: possible therapeutic and diagnostic tools in transplantation,” Front. Immunology 5:555, 2014.]

For example, heart transplant rejection is closely correlated with upregulated blood concentrations of miR-10a, miR-31, miR-92a, and miR-155. Kidney rejection has been correlated with upregulated levels of miR-142, miR-450b-5p, miR142-3p, miR-876-3p, and miR-106b, and with downregulated levels of miR508-3p, miR-148b, miR-324-5p, and miR-98. Liver transplant rejection has been correlated with upregulated levels of miR-122, miR-192, and miR-146a. [Eur. Heart J., Dec. 1, 2014; 35(45):3194-2021

MiRNA concentrations are also known to correlate with various cardiac disorders. For example, overexpressed miR-212, and miR-132 are indicative of cardiomyocyte hypertrophy [Bang, et al., “Cardiac fibroblast-derived microRNA passenger strand-enriched exosomes mediate cardiomyocyte hypertrophy,” J. Clin. Invest. 124:2136-2146, 2014,] which presents if congestive problems prompt heart enlargement. In patients experiencing heart failure, miR-423-5p and miR-18b are upregulated. miRNA expression has been extensively studied in relation to acute myocardial infarction (“AMI”). Studies have shown elevated levels of miR-208a in cardiac muscle within 1 to 3 hours of the event. miR-208b is substantially upregulated in cardiac and skeletal muscle, along with miR-133a and miR-1, while miR-499-5p presents in plasma around 24 hours after an AMI. After an AMI, miR-34a, miR-145, miR-199a-3p, miR-590-3p, miR-212, and miR-132 will be upregulated as the cardiac muscle is repaired post-infarction. [Khan, M., et al., Embryonic stem cell-derived exosomes promote endogenous repair mechanisms and enhance cardiac function following myocardial infarction,” Circ. Res. 117:52-64, 2015.] Cardiac tissue response to aerobic exercise can be registered by measuring upregulated levels of milt-27, and downregulated levels of miR-143. Genetic predisposition to hypercholesterolemia is associated with upregulated miR-223, and atherosclerosis is correlated with upregulated miR-150 and miR-135a, with downregulated miR-147.

Various levels of brain injury may also change serum miRNA levels. For instance, severe traumatic brain injury (“TBI”) may be accompanied by downregulated serum concentrations of milt-16 and miR-92a, and upregulated miR-765. Mild TBI, by contrast, will show upregulated miR-16 and miR-92a and no change to miR-765. [Jeter, et al., 2011]

Various other diseases and conditions may also be indicated by a specific miRNA profile. For example Parkinson's Disease is correlated with upregulated levels of miR-1826, miR-450b-3p, miR-505, and miR-626. Alzheimer's Disease is indicated by upregulated miR-34a, miR-181b, miR-29b, and miR-15a. Type II diabetes mellitus is correlated with downregulated miR-126, miR-15a, miR-29b, and miR-223, and upregulated miR-28-3p. Finally, ovulation is correlated with upregulated miR-132 and miR-212.

The above examples are representative only, and are not comprehensive or limiting in any manner. Other nucleotide correlations with diseases and conditions may be used to provide meaningful information to a user by a biofluid sensing device. For example, physiological reactions to toxic substances, organ failure, injuries, muscle tears, or psychiatric disorders may have correlations with changes in nucleotide concentrations, or ratios of such concentrations, that may be detectible with a properly configured biofluid sensing device. Such applications are within the spirit of the present disclosure.

This has been a description of the disclosed invention along with a preferred method of practicing the disclosed invention, however the invention itself should only be defined by the appended claims.

1.-40. (canceled)

41. A method of determining whether a biofluid sensing device is being worn on a target individual's skin, comprising:
selecting a biofluid nucleotide identification metric, wherein the identification metric is one of the following: a first biofluid nucleotide concentration; a ratio between the first nucleotide concentration and a second biofluid nucleotide concentration; a ratio among a plurality of biofluid nucleotide concentrations; a biofluid nucleotide concentration trend; or a biofluid nucleotide ratio trend;
measuring at least one identification metric on a device wearer;
comparing the identification metric with a known information set for the target individual; and
calculating an identification probability estimate that the device wearer is the target individual.

42. The method of claim 41, further comprising constructing an identification profile for the device wearer, wherein the profile includes at least one identification metric measurement.

43. The method of claim 41, wherein the information set comprises a nucleotide signature for the target individual, and wherein the nucleotide signature includes one or more identification metric values from the individual.

44. The method of claim 43, wherein the nucleotides include at least one of the following: microDNA, eccDNA, DNA fragments, microRNA, RNA fragments, and peptides.

45. The method of claim 41 wherein the identification probability estimate is calculated using data from at least one of the following secondary biometric sensors: a fingerprint scanner, a retina scanner, an accelerometer, a gait analysis sensor, an impedance respiratory monitor, a heart rate monitor, a galvanic skin response sensor, and a pulse oximetry sensor.

46. The method of claim 41, further including determining whether the device is in contact with the wearer's skin.

47. A method of determining whether a biofluid sensing device wearer has a physiological condition, comprising:
selecting a biofluid nucleotide metric having relevance to the physiological condition, wherein the metric is one of the following: a first biofluid nucleotide concentration; a ratio between the first nucleotide concentration and a second biofluid nucleotide concentration; a ratio among a plurality of biofluid nucleotide concentrations; a biofluid nucleotide concentration trend; or a biofluid nucleotide ratio trend;
measuring one or more nucleotide metric on the device wearer;
comparing the identification metric with a known information set for the physiological condition; and
calculating a probability estimate that the device wearer has the physiological condition.

48. The method of claim 47 wherein the nucleotides include one or more of the following: microDNA, eccDNA, DNA fragments, microRNA, RNA fragments, and peptides.

49. The method of claim 47, wherein the information set includes a nucleotide signature for the physiological condition, and wherein the signature includes one or more biofluid identification metric measurements from an individual having the physiological condition.

50. The method of claim 49, wherein the nucleotide signature further includes information about the device wearer.

51. The method of claim 47, further comprising measuring a molarity of one or more housekeeping microRNAs in the biofluid sample.

52. The method of claim 47, wherein the physiological condition is one of the following: chronic inflammation; cancer; general immune response; specific immune response;
bacterial infection; viral infection; autoimmune disorder; organ transplant rejection; cardiac disorder; brain injury; Parkinson's disease; Alzheimer's disease; and Type II diabetes mellitus.

53. A biofluid sensing device capable of measuring concentrations of nucleotides in a biofluid sample, and configured to be placed on a device wearer's skin, comprising:
an analyte-specific sensor to provide one or more measurements of one or more nucleotides in the biofluid sample;
a biofluid sample concentration channel having a first end and a second end, wherein the biofluid sample concentrates with respect to the at least one nucleotide as the sample moves from the first end to the second end;
a selectively permeable lysing membrane, wherein the lysing membrane is closer to the first end of the concentration channel than the sensor;
a component for lysing a nucleotide protective complex chosen from one of the following: an electrode with a power controller placed in electrical communication with the lysing membrane; and a lysing agent placed in fluid communication with the lysing membrane; and
a biofluid sample concentration membrane.

54. The device of claim 53 wherein the one or more nucleotides is one of the following: microDNA, eccDNA, DNA fragments, microRNA, RNA fragments, and peptides.

55. The device of claim 53 wherein the power controller periodically energizes the lysing membrane.

56. The device of claim 53 wherein the lysing membrane contains pores to filter out RNase molecules and nucleotide protective complexes.

57. The device of claim 53 wherein one or more of the measurements is a molarity of a housekeeping miRNA.

58. The device of claim 53 wherein the device further includes a skin contact detector.

59. The device of claim 53 wherein the at least one sensor measurement is used to construct a data set correlated with the wearer within a probability range.

60. The device of claim 59 wherein the data set is comprised of one or more of the following metrics: a first biofluid nucleotide concentration; a ratio between the first nucleotide concentration and a second biofluid nucleotide concentration; a ratio among a plurality of biofluid nucleotide concentrations; a biofluid nucleotide concentration trend; or a biofluid nucleotide ratio trend.
